SEQUENCE LISTING (8 sequences)

SEQ ID NO: 1 - protein, 9 aa, Artificial Sequence
  Description of Artificial Sequence  Synthetic Peptide
  MOD_RES (1)..(9): X = any positively charged amino acid
XZXZZZXZX

SEQ ID NO: 2 - protein, 10 aa, Artificial Sequence
  Description of Artificial Sequence  Synthetic Peptide
  MOD_RES (1)..(10): X = any positively charged amino acid
XZXZZZZXZX

SEQ ID NO: 3 - protein, 11 aa, Artificial Sequence
  Description of Artificial Sequence  Synthetic Peptide
  MOD_RES (1)..(11): X = any positively charged amino acid
XZXZZZZZXZX

SEQ ID NO: 4 - protein, 12 aa, Artificial Sequence
  Description of Artificial Sequence  Synthetic Peptide
  MOD_RES (1)..(12): X = any positively charged amino acid
XZXZZZZZZXZX

SEQ ID NO: 5 - protein, 13 aa, Artificial Sequence
  Description of Artificial Sequence  Synthetic Peptide
  MOD_RES (1)..(13): X = any positively charged amino acid
XZXZZZZZZZXZX

SEQ ID NO: 6 - protein, 11 aa, Artificial Sequence
  Description of Artificial Sequence  Synthetic Peptide
KLKLLLLLKLK

SEQ ID NO: 7 - DNA, 20 bp, Artificial Sequence
  MOD_BASE (9)..(9): n = i
  Description of Artificial Sequence  Synthetic Primer
tccatgacnttcctgatgct

SEQ ID NO: 8 - protein, 26 aa, Artificial Sequence
  Description of Artificial Sequence  Synthetic Peptide
ICICICICICICICICICICICICIC

(19) United States
(12) Patent Application Publication
(10) Pub. No.: US 2007/0041998 A1
(43) Pub. Date: Feb. 22, 2007

(54) Use of alum and a th1 immune response inducing adjuvant for enhancing immune responses

(75) Inventors: Michael Buschle, Perchtoldsdorf (AT); Karen Lingnau, Vienna (AT)

Correspondence Address: FULBRIGHT & JAWORSKI L.L.P., 600 CONGRESS AVE., SUITE 2400, AUSTIN, TX, 78701, US

(73) Assignee: Intercell AG, Vienna (AT)

(21) Appl. No.: 10/550,820

(22) PCT Filed: Mar. 22, 2004
(86) PCT No.: PCT/EP04/03029

§ 371 Date: Sep. 23, 2005

(30) Foreign Application Priority Data
Mar. 24, 2003 (EP) 034500728

(51) Int. Cl. A61K 39/12; A61K 39/21; A61K 39/145; A61K 39/29; A61K 39/02

(52) U.S. Cl. 424/204.1; 424/208.1; 424/209.1; 424/225.1; 514/44; 424/234.1

(57) ABSTRACT

The invention relates to the use of Alum for the preparation of a drug for enhancing an antigen-specific type 1 immune response against an antigen in the presence of a type 1 inducing adjuvant.

The present invention relates to a use of Alum for enhancing immune responses.

Host protection from invading pathogens involves cellular and humoral effectors and results from the concerted action of both non-adaptive (innate) and adaptive (acquired) immunity. The latter is based on specific immunological recognition mediated by receptors, is a recent acquisition of the immune system, and is present only in vertebrates. The former evolved before the development of adaptive immunity, consisting of a variety of cells and molecules distributed throughout the organism with the task of keeping potential pathogens under control.

B and T lymphocytes are the mediators of acquired antigen-specific adaptive immunity, including the development of immunological memory, which is the main goal of creating a successful vaccine. Antigen presenting cells (APCs) are highly specialized cells that can process antigens and display their processed fragments on the cell surface together with molecules required for lymphocyte activation. This means that APCs are very important for the initiation of specific immune reactions. The main APCs for T lymphocyte activation are dendritic cells (DCs), macrophages, and B cells, whereas the main APCs for B cells are follicular dendritic cells. In general DCs are the most powerful APCs in terms of initiation of immune responses stimulating quiescent naive and memory B and T lymphocytes.

The natural task of APCs in the periphery (e.g. DCs or Langerhans cells) is to capture and process antigens, thereby being activated they start to express lymphocyte co-stimulatory molecules, migrate to lymphoid organs, secrete cytokines and present antigens to different populations of lymphocytes, initiating antigen-specific immune responses. They not only activate lymphocytes, under certain circumstances, they also tolerize T cells to antigens.

Antigen recognition by T lymphocytes is major histocompatibility complex (MHC)-restricted. A given T lymphocyte will recognize an antigen only when the peptide is bound to a particular MHC molecule. In general, T lymphocytes are stimulated only in the presence of self MHC molecules, and antigen is recognized only as peptides bound to self MHC molecules. MHC restriction defines T lymphocyte specifity in terms of the antigen recognized and in terms of the MHC molecule that binds its peptide fragment.

Intracellular and extracellular antigens present quite different challenges to the immune system, both in terms of recognition and of appropriate response. Presentation of antigens to T cells is mediated by two distinct classes of molecules—MHC class I (MHC-I) and MHC class II (MHC-II), which utilize distinct antigen processing pathways. Mainly one could distinguish between two major antigen processing pathways that have evolved. Peptides derived from intracellular antigens are presented to CD8⁺ T cells by MHC class I molecules, which are expressed on virtually all cells, while extracellular antigen-derived peptides are presented to CD4⁺ T cells by MHC-II molecules. However, there are certain exceptions to this dichotomy. Several studies have shown that peptides generated from endocytosed particulate or soluble proteins are presented on MHC-I molecules in macrophages as well as in dendritic cells. Therefore APCs like dendritic cells sitting in the periphery, exerting high potency to capture and process extracellular antigens and presenting them on MHC-I molecules to T lymphocytes are interesting targets in pulsing them extracellularily with antigens in vitro and in vivo.

The important and unique role of APCs, including stimulating activity on different types of leukocytes, is reflecting their central position as targets for appropriate strategies in developing successful vaccines. Theoretically one way to do so is to enhance or stimulate their natural task, the uptake of antigen(s). Once pulsed with the appropriate antigens the vaccine is directed against, APCs should start to process the uptaken antigen(s), thereby being activated, expressing lymphocyte co-stimulatory molecules, migrating to lymphoid organs, secreting cytokines and presenting antigens to different populations of lymphocytes thereby initiating immune responses.

Activated T cells generally secrete a number of effector cytokines in a highly regulated fashion, e.g. interleukin 2 (IL-2), IL-4, IL-5, IL-10 and interferon-γ (IFN-γ). The functional detection of cytotoxic T lymphocyte responses to specific antigens (e.g. tumor antigens, in general antigens administered in a vaccine) is commonly monitored by an ELISpot assay (enzyme-linked immunospot assay), a technique analyzing cytokine production at the single cell level. In the present invention an ELISpot assay for the cellular immunity (type 1 immune response) promoting cytokine IFN-γ is used to monitor successful antigen-specific T cell activation. Furthermore, the cytokine IL-4 is determined as an indicator for a type 2 response, usually involved in promoting strong humoral responses. In addition, the humoral immune response was determined by ELISA (IgG1 as indicator for a type 2 response, IgG2b as indicator for a type 1 response).

It has previously been shown that polycations efficiently enhance the uptake of MHC class I-matched peptides into tumor cells, a peptide or protein pulsing process which was called “TRANSloading”. Furthermore, it has been shown that polycations are able to “TRANSload” peptides or proteins into antigen presenting cells in vivo as well as in vitro. In addition, co-injection of a mixture of poly-L-arginine or poly-L-lysine together with an appropriate peptide as a vaccine protects animals from tumor growth in mouse models. This chemically defined vaccine is able to induce a high number of antigen/peptide-specific T cells. That was shown to be at least partly attributable to an enhanced uptake of peptides into APCs mediated by the polycation indicating that APCs when pulsed in vivo with antigens can induce T cell-mediated immunity to the administered antigen.

As opposed to adaptive immunity, which is characterized by a highly specific but relatively slow response, innate immunity is based on effector mechanisms that are triggered by differences in the structure of microbial components relative to the host. These mechanisms can mount a fairly rapid initial response, which mainly leads to neutralization of the noxious agents. Reactions of innate immunity are the only defense strategy of lower phyla and have been retained in vertebrates as a first line host defense before the adaptive immune system is mobilized.

In higher vertebrates the effector cells of innate immunity are neutrophils, macrophages, and natural killer cells and probably also dendritic cells, whereas the humoral components in this pathway are the complement cascade and a variety of different binding proteins.

A rapid and effective component of innate immunity is the production of a large variety of microbicidal peptides with a length of usually between about 12 and about one hundred amino acid residues. Several hundred different antimicrobial peptides have been isolated from a variety of organisms, ranging from sponges, insects to animals and humans, which points to a wide-spread distribution of these molecules. Antimicrobial peptides are also produced by bacteria as antagonistic substances against competing organisms.

Two major subsets of CD4⁺ T cells (T-helper 1 (Th1) and T-helper 2 (Th2)) have been identified in mouse and human, based on their secretion of different cytokine profiles and their different effector functions. Th1 cells are mainly involved in the generation of so called type 1 immune responses, which are typically characterised by the induction of delayed-type hypersensitivity responses, cell-mediated immunity, immunoglobulin class switching to IgG2a/IgG2b and secretion of i.a. Interferon-γ. In contrast, Th2 cells are involved in the generation of so called type 2 responses, which are characterised by the induction of humoral immunity by activating B cells, leading to antibody production including class switching to IgG₁ and IgE. Type 2 responses are also characterized by the secretion of the following cytokines: IL-4, IL-5, IL-6 and IL-10.

In most situations, the type of response induced (type 1 or type 2) has a significant impact on the protective efficacy of a vaccine. Alternative adjuvants tend to favor specific types of responses. However, adjuvant selection is complicated by functional unpredictabilities and also by commercial constraints and availability.

Aluminum salts (e.g. Aluminum hydroxide (Alum) (Römpp, 10^{th} Ed. pages 139/140), Aluminum phosphate) are currently used as a vaccine adjuvant in almost all available human vaccines [1]. However, aluminum salts were shown to increase in humans, as well as in animals, exclusively a shift to type 2 responses (cellular: IL-4 production, humoral: IgG₁, IgE) [2]. The inability of aluminum salts to elicit type 1 cell-mediated immune responses (cellular: IFN-γ production, humoral: IgG₂) is a major limitation of its use as adjuvant. Particularly for vaccines against intracellular viral and bacterial infections, the lack of cytotoxic T cell responses is fatal.

Therefore, a need exists to provide improved vaccines which show a type 1 directed immune response or vaccines which allow—in addition to a type 2 response—also a type 1 shift of the immune reaction. Moreover, vaccines already available should be provided in an improved form which allows the induction of a type 1 response.

The present invention therefore provides novel pharmaceutical compositions, comprising:

an antigen,

a type 1 adjuvant and

Alum,

with the proviso that the type 1 inducing adjuvant is not an oligodeoxynucleotide containing a CpG motif (an unmethylated CpG motif).

It has been surprisingly shown with the present invention that Alum can enhance the type 1 potency of a given type 1 inducing adjuvant in a vaccine (and leaving type 2 potency generally unaffected). This could not be expected from the prior art because Alum was regarded as being exclusively type 2 directed. Indeed, the immune reaction of a given antigen, if applied alone and in combination with Alum, is significantly enhanced with respect to the type 1 reaction (whereby type 2 activity is conserved) if Alum is present. Therefore, any (even slightly) positive or even neutral effect on the type 1 response of Alum was not foreseeable by the prior art.

The present invention is based on the fact that alum can efficiently enhance the type 1 response induced by a vaccine, if a type 1 inducing adjuvant is already present in the vaccine. If such a type 1 inducing adjuvant is not present, enhancement of type 1 responses does not occur.

Alum, as meant herein includes all forms of Al³⁺ based adjuvants used in human and animal medicine and research. Especially, it includes all forms of aluminum hydroxide as defined in Römpp, 10^{th} Ed. pages 139/140, gel forms thereof, aluminum phosphate, etc.

With the present invention, a clear improvement of the cellular type 1 response is provided (IFN-g), without reduced IgG responses.

The antigen to be used according to the present invention is not critical, however, if pronounced (or exclusive) type 1 responses should be specifically necessary, T cell epitopes (see introduction above) are preferred as antigens. Preferably the antigen is a viral, parasitic or bacterial antigen. In the example section the present invention is proven in principle with hepatitis viral antigens, namely with the hepatitis B surface antigen, which are preferred antigens according to the present invention.

Of course, the pharmaceutical preparation may also comprise two or more antigens depending on the desired immune response. The antigen(s) may also be modified so as to further enhance the immune response.

Preferably, proteins or peptides derived from viral or bacterial pathogens, from fungi or parasites, as well as tumor antigens (cancer vaccines) or antigens with a putative role in autoimmune disease are used as antigens (including derivatized antigens like glycosylated, lipidated, glycolipidated or hydroxylated antigens). Furthermore, carbohydrates, lipids or glycolipids may be used as antigens themselves. The derivatization process may include the purification of a specific protein or peptide from the pathogen, the inactivation of the pathogen as well as the proteolytic or chemical derivatization or stabilization of such a protein or peptide. Alternatively, also the pathogen itself may be used as an antigen. The antigens are preferably peptides or proteins, carbohydrates, lipids, glycolipids or mixtures thereof.

According to a preferred embodiment, T cell epitopes are used as antigens. Alternatively, a combination of T cell epitopes and B cell epitopes may also be preferred.

Also mixtures of different antigens are of course possible to be used according to the present invention. Preferably, proteins or peptides isolated from a viral or a bacterial pathogen or from fungi or parasites (or their recombinant counterparts) are used as such antigens (including derivatized antigens or glycosylated or lipidated antigens or polysaccharides or lipids). Another preferred source of antigens are tumor antigens. Preferred pathogens are selected from human immunodeficiency virus (HIV), hepatitis A and B viruses, hepatitis C virus (HCV), human papilloma virus (HPV), rous sarcoma virus (RSV), Epstein Barr virus (EBV) Influenza virus, Rotavirus, Staphylococcus aureus, Chlamydia pneumonias, Chlamydia trachomatis, Mycobacterium tuberculosis, Streptococcus pneumonias, Bacillus anthracis, Vibrio cholerae, Plasmodium sp. (Pl. falciparum, Pl. vivax, etc.), Aspergillus sp. or Candida albicans. Antigens may also be molecules expressed by cancer cells (tumor antigens). The derivation process may include the purification of a specific protein from the pathogen/cancer cells, the inactivation of the pathogen as well as the proteolytic or chemical derivatization or stabilisation of such a protein. In the same way also tumor antigens (cancer vaccines) or autoimmune antigens may be used in the pharmaceutical composition according to the present invention. With such compositions a tumor vaccination or a treatment for autoimmune diseases may be performed.

In the case of peptide antigens the use of peptide mimotopes/agonists/superagonists/antagonists or peptides changed in certain positions without affecting the immunologic properties or non-peptide mimotopes/agonists/superagonists/antagonists is included in the current invention. Peptide antigens may also contain elongations either at the carboxy or at the amino terminus of the peptide antigen facilitating interaction with the polycationic compound(s) or the immunostimulatory compound(s). For the treatment of autoimmune diseases peptide antagonists may be applied.

Antigens may also be derivatized to include molecules enhancing antigen presentation and targeting of antigens to antigen presenting cells.

In one embodiment of the invention the pharmaceutical composition serves to confer tolerance to proteins or protein fragments and peptides which are involved in autoimmune diseases. Antigens used in this embodiments serve to tolerize the immune system or downregulate immune responses against epitopes involved in autoimmune processes.

Preferably, the antigen is a peptide consisting of 5 to 60, preferably 6 to 30, especially 8 to 11, amino acid residues (e.g. a naturally isolated, recombinantly or chemically produced fragment of a pathogen-derived protein, especially with an immunogenic epitope). Antigens of this length have been proven to be especially suitable for T cell activation. The antigens can further be coupled with a tail, e.g. according to WO 01/78767, U.S. Pat. No. 5,726,292 or WO 98/01558.

The structural nature of the type 1 inducing adjuvant (Immunizer) to be combined with Alum has been shown to be of low relevance for the present invention; the synergistic effect is almost exclusively connected to the functional type 1 directing ability of the adjuvant (Immunizer) or adjuvant (Immunizer) mixture when combined with Alum. Preferably the type 1 inducing adjuvant (Immunizer) is selected from the group consisting of a polycationic polymer, lipid particle emulsions, especially MF59, stable formulations of squalene and pluronid polymers and the threonyl analog of muramyl dipeptide (syntex adjuvant formulation (SAF), monophosphoryl Lipid A (MPL), saponins, especially QS21, an immunstimulatory oligodeoxynucleotide (ODN), with the proviso that the immunostimulatory oligodeoxynucleotide is not an oligodeoxynucleotide containing a CpG motif, and combinations thereof.

It has been shown previously (WO 02/13857) that naturally occurring, cathelicidin-derived antimicrobial peptides or derivatives thereof have an immune response stimulating activity and therefore constitute highly effective type 1 inducing adjuvants (Immunizers). Main sources of antimicrobial peptides are granules of neutrophils and epithelial cells lining the respiratory, gastro-intestinal and genitourinary tracts. In general they are found at those anatomical sites most exposed to microbial invasion, are secreted into internal body fluids or stored in cytoplasmic granules of professional phagocytes (neutrophils).

In the WO 02/32451 a type 1 inducing adjuvant (Immunizer) that is able to strongly enhance the immune response to a specific co-administered antigen and therefore constitutes a highly effective adjuvant is disclosed. The adjuvant (Immunizer) according to the WO 02/32451 is a peptide comprising a sequence R₁-XZXZ_{N}XZX-R₂, whereby N is a whole number between 3 and 7, preferably 5, X is a positively charged natural and/or non-natural amino acid residue, Z is an amino acid residue selected from the group consisting of L, V, I, F and/or W, and R₁ and R₂ are selected independently one from the other from the group consisting of —H, —NH₂, —COCH₃, —COH, a peptide with up to 20 amino acid residues or a peptide reactive group or a peptide linker with or without a peptide; X—R₂ may also be an amide, ester or thioester of the C-terminal amino acid residue. A specifically preferred peptide is KLKLLLLLKLK.

Besides naturally occurring antimicrobial peptides, synthetic antimicrobial peptides have been produced and investigated. The synthetic antimicrobial peptide KLKLLLLLKLK-NH₂ was shown to have significant chemotherapeutic activity in Staphylococcus aureus-infected mice; human neutrophils were activated to produce the superoxide anion (O₂⁻) via cell surface calreticulin. The exact number and position of K and L was found to be critical for the antimicrobial activity of the synthetic peptide (Nakajima, Y. (1997); Cho, J-H. (1999)).

The polycationic polymer(s) or compound(s) to be used as type 1 stimulators according to the present invention may be any polycationic compound which shows the characteristic effect according to the WO 97/30721 (and which is, of course, not the antigen for which immunisation is sought for). Preferred polycationic compounds are selected from basic polypeptides, organic polycations, basic polyaminoacids or mixtures thereof. These polyaminoacids should have a chain length of at least 4 amino acid residues. Especially preferred are substances containing peptidic bounds, like polylysine, polyarginine and polypeptides containing more than 20%, especially more than 50% of basic amino acids in a range of more than 8, especially more than 20, amino acid residues or mixtures thereof. Other preferred polycations and their pharmaceutical compositons are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be polycationic anti-bacterial microbial peptides. These (poly)peptides may be of prokaryotic or eukaryotic origin or may be produced chemically or recombinantly. Peptides may also belong to the class naturally occurring antimicrobial peptides. Such host defense peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Furthermore, also neuroactive compounds, such as (human) growth hormone (as described e.g. in WO01/24822) may be used as Th1 immunostimulants (immunisers).

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelicidin, especially mouse, bovine or especially human cathelicidins and/or cathelicidins. Related or derived cathelicidin substances contain the whole or parts of the cathelicidin sequence with at least 15-20 amino acid residues. Derivations may include the substitution or modification of the natural amino acids by amino acids which are not among the 20 standard amino acids. Moreover, further cationic residues may be introduced into such cathelicidin molecules. These cathelicidin molecules are preferred to be combined with the antigen/vaccine composition according to the present invention. However, these cathelin molecules surprisingly have turned out to be also effective as an adjuvant for a antigen without the addition of further adjuvants. It is therefore possible to use such cathelicidin molecules as efficient adjuvants in vaccine formulations with or without further immunactivating substances.

According to a significantly preferred embodiment of the present invention, the pharmaceutical composition comprises an immunostimulatory ODN selected from the group consisting of a deoxynucleotide comprising (one or more) deoxyinosine and/or deoxyuridine residues; a deoxynucleotide comprising at least one 2′deoxycytosine-monophosphate or -monothiophosphate 3 adjacent to a 2′deoxyinosine-monophosphate or -monothiophosphate, especially a deoxyinosine-deoxycytosine 26-mer; and an ODN based on inosine and cytidine.

The pharmaceutical composition according to the present invention may also contain a mixture of more than one type 1 inducing adjuvant (Immunizer), i.e. a type 1 inducing adjuvant (Immunizer) composition. In this type 1 inducing adjuvant (Immunizer) composition it is preferred to additionally provide a (one or more) polycationic polymer selected from the group consisting of a synthetic peptide containing at least 2 KLK motifs separated by a linker of 3 to 7 hydrophobic amino acids, preferably a peptide with the sequence KLKLLLLLKLK; a polycationic peptide, especially polyarginine, polylysine and an antimicrobial peptide, especially a cathelicidin-derived antimicrobial peptide. As stated above, it is specifically preferred to combine such peptidic immunisers with the above mentioned significantly preferred selected oligodeoxynucleotides (I- or U-ODNs). Such I- and U-ODNs are specifically characterised as an immunostimulatory oligodeoxynucleic acid molecule (ODN) having the structure according to the formula (I) 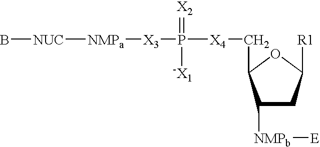
wherein
R1 is selected from hypoxanthine and uracile,
any X is O or S,
any NMP is a 2′ deoxynucleoside monophosphate or monothiophosphate, selected from the group consisting of deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphate,
NUC is a 2′ deoxynucleoside, selected from the group consisting of deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine,
a and b are integers from 0 to 100 with the proviso that a+b is between 4 and 150, and
B and E are common groups for 5′ or 3′ ends of nucleic acid molecules.

According to another aspect, the present invention also relates to the use of Alum for the preparation of a drug for enhancing an antigen-specific type 1 immune response against an antigen in the presence of a type 1 inducing adjuvant (Immunizer).

More specifically, Alum is used according to the present invention for the preparation of a vaccine with enhanced type 1 inducing activity.

The present invention also relates to the use of the combination of a type 1 inducing adjuvant (Immunizer) and Alum as a type 1 inducing adjuvant (Immunizer). Improved type 1 inducing adjuvants (type 1 adjuvant compositions) are therefore provided by the present invention.

According to the present invention a type 1 inducing adjuvant (Immunizer) composition is provided which comprises a type 1 inducing adjuvant (Immunizer) and Alum, with the proviso that the type 1 inducing adjuvant is not an oligodeoxynucleotide containing a CpG motif (an unmethylated ODN with CpG motif(s)).

An adjuvant (Immunizer), which based on a combination of a cationic poly-amino acid and a synthetic ODN, is specifically preferred to be combined with Alum according to the present application to induce as a vaccine adjuvant potent antigen-specific type 1 immune responses.

According to the present invention, any given vaccine containing Alum as an adjuvant can effectively be improved by the addition of the selected type 1 inducing adjuvant (Immunizer) (composition) according to the present invention, especially by the addition of an I- and/or a U-ODN, optionally admixed with a polycationic peptide compound (a peptidic (type 1) adjuvant (Immunizer)).

The antigen may be mixed with the adjuvant (Immunizer) (composition) according to the present invention or otherwise specifically formulated e.g. as liposome, retard formulation, etc.

The present invention is especially beneficial if the combined medicament is administered, e.g. subcutaneously, intravenously, intranasally, oral, intramusculary, intradermally or transdermally. However, other application forms, such as parenteral or topical application, are also suitable for the present invention.

The invention will be described in more detail by the following examples and figures, but the invention is of course not limited thereto.

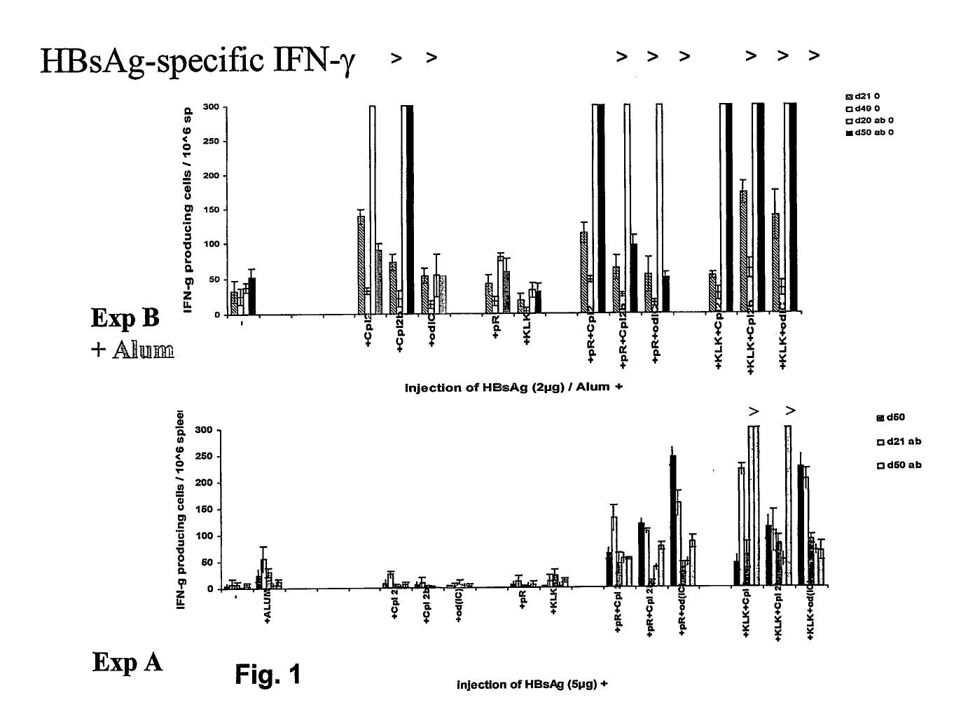

FIG. 1 shows the induction of a HBsAg-specific cellular type 1 response after injection of HBsAg alone or in combination with Alum and other adjuvants (Immunizers) (HBsAg-specific IFN-γ production).

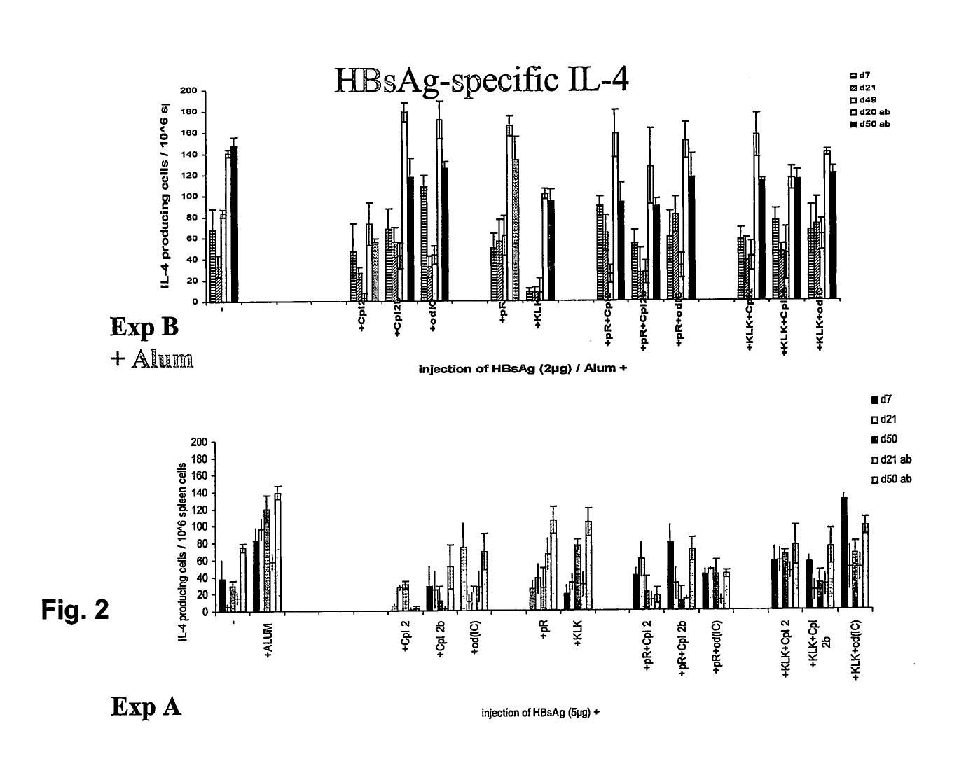

FIG. 2 shows the induction of a HBsAg-specific cellular type 2 response after injection of HBsAg alone or in combination with Alum and other adjuvants (Immunizers) (HBsAg-specific IL-4 production).

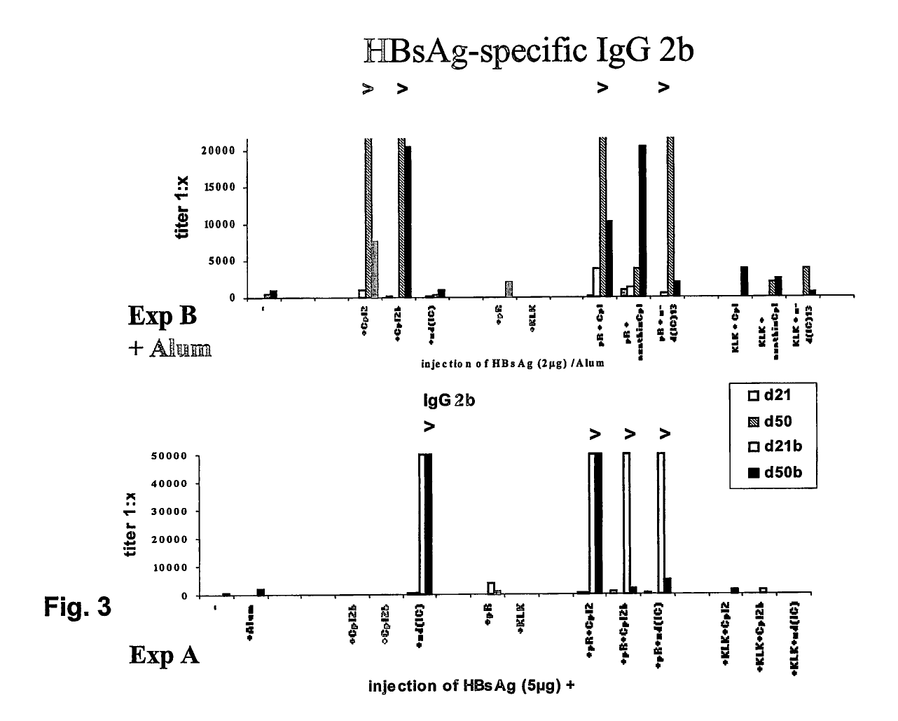

FIG. 3 shows the induction of a HBsAg-specific humoral type 1 response after injection of HBsAg alone or in combination with Alum and other adjuvants (Immunizers) (HBsAg-specific IgG_{2b} titer).

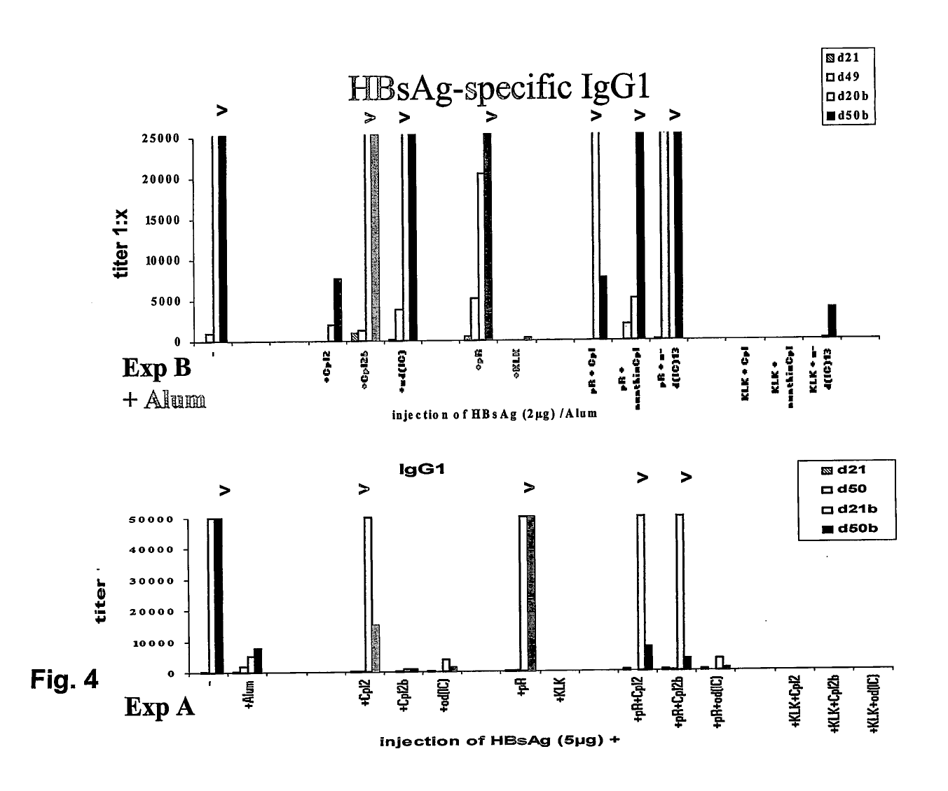

FIG. 4 shows the induction of a HBsAg-specific humoral type 2 response after injection of HBsAg alone or in combination with Alum and other adjuvants (Immunizers) (HBsAg-specific IgG₁ titer).

### EXAMPLES

Herein, an example is provided, which shows that upon co-injection of the Hepatitis B surface Antigen (HBsAg), various type 1 inducing adjuvants (Immunizers) and Alum the type 1 response induced by the type 1 inducing adjuvants (Immunizers) is strongly increased at least after boost when compared to injection of HBsAg/Immunizer alone. However, the Alum-induced type 2 response is not affected.

Materials and Methods:

| | |
|---|---|
| Mice | C57B1/6 (Harlan-Winkelmann, Germany); low |
| | responder mice for HbsAg-specific immune |
| | responses |
| | 5 mice/group/timepoint |
| Antigen | Hepatitis B surface antigen (HBsAg) |
| | dose: 5 μg/mouse |
| poly-L-arginine | poly-L-arginine with an average degree of |
| | polymerisation of 43 arginine residues; Sigma |
| | chemicals |
| | dose: 100 μg/mouse |
| KLK | KLKLLLLLKLK-COOH was synthesized by MPS |
| | (Multiple Peptide System, USA) |
| | Dose: 168 μg/mouse |
| I-ODN 2 | thiophosphate substituted ODNs containing |
| | deoxyinosines: 5′tcc atg aci ttc ctg atg ct |
| | 3′ were synthesized by Purimex Nucleic Acids |
| | Technology, Göttingen |
| | Dose: 5 nmol/mouse |
| I-ODN 2b | ODNs containing deoxyinosines: 5′tcc atg aci |
| | ttc ctg atg ct 3′ were synthesized by Purimex |
| | Nucleic Acids Technology, Göttingen |
| | Dose: 5 nmol/mouse |
| o-d(IC)₁₃ | ODN 5′ICI CIC ICI CIC ICI CIC ICI CIC IC3′ |
| | was synthesized by Purimex Nucleic Acids |
| | Technology, Göttingen |
| | Dose: 5 nmol/mouse |

Exp A:

| | |
|---|---|
| 1. | HBsAg |
| 2. | HBsAg + Alum |
| 3. | HBsAg + I-ODN 2 |
| 4. | HBsAg + I-ODN 2b |
| 5. | HBsAg + o-d(IC)₁₃ |
| 6. | HBsAg + pR |
| 7. | HBsAg + KLK |
| 8. | HBsAg + pR + I-ODN 2 |
| 9. | HBsAg + pR + I-ODN 2b |
| 10. | HBsAg + pR + o-d(IC)₁₃ |
| 11. | HBsAg + KLK + I-ODN 2 |
| 12. | HBsAg + KLK + I-ODN 2b |
| 13. | HBsAg + KLK + o-d(IC)₁₃ |

Exp B:

| | |
|---|---|
| 1. | HbsAg/Alum |
| 2. | HbsAg/Alum + I-ODN 2 |
| 3. | HbsAg/Alum + I-ODN 2b |
| 4. | HbsAg/Alum + o-d(IC)₁₃ |
| 5. | HbsAg/Alum + pR |
| 6. | HBsAg/Alum + KLK |
| 7. | HbsAg/Alum + pR + I-ODN 2 |
| 8. | HbsAg/Alum + pR + I-ODN 2b |
| 9. | HbsAg/Alum + pR + o-d(IC)₁₃ |
| 10. | HBsAg/Alum + KLK + I-ODN 2 |
| 11. | HBsAg/Alum + KLK + I-ODN 2b |
| 12. | HbsAg/Alum + KLK + o-d(IC)₁₃ |

On day 0 and day 56 mice were injected subcutaneously into the right flank with a total volume of 100 μl/mouse containing the above mentioned compounds. The analysis of the immune response was performed at (day 7) day 21 and day 50 after first and second injection, respectively. Spleen cells of five mice per group per time point were restimulated ex vivo with 10 μg/ml HBsAg and ELISPOT assays were performed in order to analyse the HBsAg-specific IFN-γ (type 1 immune response) as well as IL-4 (type 2 immune response) production. Furthermore, serum was taken at the indicated time points and the HBsAg-specific IgG_{2b} (type 1 immune response) as well as IgG₁ (type 2 immune response) titers were determined.

Results:

FIG. 1: Induction of a HBsAg-Specific Cellular Type 1 Response (HBsAg-specific IFN-γ Production)

HBsAg injected alone or in combination with Alum induces no or only very low levels of IFN-γ, whereas upon injection of HBsAg combined with the different Immunizers (pR/ODN, KLK/ODN) an HBsAg-specific IFN-γ production is induced which can be further increased by booster vaccination (Exp. A). However, upon co-injection of HBsAg/Immunizer and Alum the induced IFN-γ production after boost is strongly increased (Exp. B).

FIG. 2: Induction of a HBsAg-Specific Cellular Type 2 Response (HBsAg-Specific IL-4 Production)

HBsAg injected in combination with Alum induces HBsAg-specific IL-4 production, which is not further affected by the co-injection of the different Immunizers (Exp. B).

FIG. 3: Induction of a Humoral Type 1 Response (HBsAg-Specific IgG2b Titer)

HBsAg injected alone or in combination with Alum induces no HBsAg-specific IgG2b, whereas upon injection of HBsAg combined with the different pR/ODN-based Immunizers potent IgG2b titers are detectable after boost (Exp. A). The co-injection of Alum has no real influence on these titers (Exp. B). Upon injection of HBsAg/KLK-ODN-based Immunizer no antibody titers are induced at all (Exp. A, B).

FIG. 4: Induction of a Humoral Type 2 Response (HBsAg-Specific IgG1 Titer)

HBsAg injected in combination with Alum induces HBsAg-specific IgG1 titer, which are not further affected by the co-injection of the pR/ODN-based Immunizer (Exp. B). Upon use of KLK-ODN-based Immunizer no antibody titers are induced at all (Exp. A, B).

### CONCLUSIONS

Compared to the injection of antigen with Immunizers, the co-injection of Immunizers with Alum induce enhanced cellular type 1 immune responses (IFN-γ), while the Alum-induced type 2 response (IL-4) is not affected. This observation makes the Immunizers very attractive in at least two ways. On the one hand, existing Alum-based vaccines can be improved by type 1 inducing Immunizers, e.g. in order to induce cell mediated type 1 responses which were lacking so far for special applications like therapeutic vaccines against viral infections. On the other hand, more potent type 1 responses can be induced in general when the combination Immunizer/Alum is used as vaccine adjuvant.

### REFERENCES

(1) Shirodkar, S., et al, 1990, Aluminum compounds used as adjuvant in vaccines, Pharm Res. 7:1282-1288
(2) Gupta, R. K. and Siber, G. R.; 1995, Adjuvants for human vaccines—current status, problems and future prospects, Vaccine 13(14) 1263-1276

1.-15. (canceled)

16. A pharmaceutical composition, comprising:
an antigen;
a type 1 inducing adjuvant that is not an oligodeoxynucleotide (ODN) containing a CpG motif; and
Alum.

17. The pharmaceutical composition of claim 16, wherein the antigen is a viral, parasitic or bacterial antigen.

18. The pharmaceutical composition of claim 17, wherein the antigen is a hepatitis viral antigen, HIV-, HPV-, or influenza antigen.

19. The pharmaceutical composition of claim 18, wherein the antigen is a hepatitis viral antigen further defined as a hepatitis A, hepatitis B, hepatitis C, or hepatitis D antigen.

20. The pharmaceutical composition of claim 16, wherein the type 1 inducing adjuvant is a polycationic polymer, lipid particle emulsion, stable formulation of squalene and pluronid polymers and threonyl analogs of MDP (syntex adjuvant formulation (SAF)), monophosphoryl Lipid A (MPL), saponin, and/or an immunostimulatory oligodeoxynucleotide (ODN) that does not contain a CpG motif.

21. The pharmaceutical composition of claim 20, wherein the type 1 inducing adjuvant is a lipid particle emulsion further defined as MF59.

22. The pharmaceutical composition of claim 20, wherein the type 1 inducing adjuvant is a saponin further defined as QS21.

23. The pharmaceutical composition of claim 20, wherein the type 1 inducing adjuvant is an immunostimulatory ODN further defined as a deoxynucleotide comprising deoxyinosine and/or deoxyuridine residues; a deoxynucleotide comprising at least one 2′deoxycytosine-monophosphate or -monothiophosphate 3′ adjacent to a 2′deoxyinosine-monophosphate or -monothiophosphate, or an ODN based on inosine and cytidine.

24. The pharmaceutical composition of claim 23, wherein the type 1 inducing adjuvant is a deoxyinosine-deoxycytosine 26-mer.

25. The pharmaceutical composition of claim 20, wherein the type 1 inducing adjuvant is a polycationic polymer further defined as a synthetic peptide containing at least 2 KLK motifs separated by a linker of 3 to 7 hydrophobic amino acids; a polycationic peptide, polylysine, or an antimicrobial peptide.

26. The pharmaceutical composition of claim 25, wherein the type 1 inducing adjuvant is a synthetic peptide with the sequence KLKLLLLLKLK.

27. The pharmaceutical composition of claim 25, wherein the type 1 inducing adjuvant is polyarginine.

28. The pharmaceutical composition of claim 25, wherein the type 1 inducing adjuvant is a cathelicidin-derived antimicrobial peptide.

29. A method of enhancing an antigen-specific type 1 immune response against an antigen comprising:
obtaining a pharmaceutical composition comprising an antigen, a type 1 inducing adjuvant that is not an oligodeoxynucleotide (ODN) containing a CpG motif, and Alum; and
administering the pharmaceutical composition to a subject;
wherein an antigen-specific type 1 immune response against antigen is enhanced in the subject.

30. The method of claim 29, wherein the antigen is a viral, parasitic or bacterial antigen.

31. The method of claim 30, wherein the antigen is a viral antigen further defined as a hepatitis viral antigen, HIV-, HPV-, or influenza antigen.

32. The method of claim 31, wherein the antigen is a hepatitis viral antigen further defined as a hepatitis A, hepatitis B, hepatitis C, or hepatitis D antigen.

33. The method of claim 29, wherein the type 1 inducing adjuvant is selected from the group consisting of a polycationic polymer, lipid particle emulsions, especially MF59, stable formulations of squalene and pluronid polymers and threonyl analogs of MDP (syntex adjuvant formulation (SAF)), monophosphoryl Lipid A (MPL), saponins, especially QS21, an immunostimulatory oligodeoxynucleotide (ODN), and combinations thereof.

34. The method of claim 29, wherein the type 1 inducing adjuvant is a lipid particle emulsion further defined as MF59.

35. The method of claim 29, wherein the type 1 inducing adjuvant is a saponin further defined as QS21.

36. The method of claim 29, wherein the type 1 inducing adjuvant is an immunostimulatory ODN further defined as a deoxynucleotide comprising deoxyinosine and/or deoxyuridine residues; a deoxynucleotide comprising at least one 2′deoxycytosine-monophosphate or -monothiophosphate 3′ adjacent to a 2′deoxyinosine-monophosphate or -monothiophosphate, or an ODN based on inosine and cytidine.

37. The method of claim 36, wherein the type 1 inducing adjuvant is a deoxyinosine-deoxycytosine 26-mer.

38. The method of claim 29, wherein the type 1 inducing adjuvant is a polycationic polymer further defined as a synthetic peptide containing at least 2 KLK motifs separated by a linker of 3 to 7 hydrophobic amino acids; a polycationic peptide, polylysine, or an antimicrobial peptide.

39. The method of claim 38, wherein the type 1 inducing adjuvant is a synthetic peptide with the sequence KLKLLLLLKLK.

40. The method of claim 38, wherein the type 1 inducing adjuvant is polyarginine.

41. The method of claim 38, wherein the type 1 inducing adjuvant is a cathelicidin-derived antimicrobial peptide.

42. The method of claim 29, wherein the subject is human.
